(19) United States
(12) Patent Application Publication
(10) Pub. No.: US 2024/0148329 A1
(43) Pub. Date: May 9, 2024

(54) DEVICES, SYSTEMS, METHODS AND COMPUTER-ACCESSIBLE MEDIUM FOR PROVIDING WIRELESS STENT-BASED INTERFACES TO THE NERVOUS SYSTEM

(71) Applicants: The Trustees of Columbia University in The City of New York, New York, NY (US); Massachusetts Institute of Technology, Cambridge, MA (US)

(72) Inventors: Kenneth L. SHEPARD, Ossining, NY (US); John William STANTON, New York, NY (US); Giovanni Talei FRANZESI, Boston, MA (US); Ed BOYDEN, Chestnut Hill, MA (US)

(21) Appl. No.: 18/507,676

(22) Filed: Nov. 13, 2023

Related U.S. Application Data
(63) Continuation of application No. PCT/US2022/029625, filed on May 17, 2022.
(60) Provisional application No. 63/189,536, filed on May 17, 2021.

(51) Int. Cl. A61B 5/00; A61B 5/07; H05K 1/03

(52) U.S. Cl. CPC A61B 5/686; A61B 5/0031; A61B 5/076; H05K 1/0393

(57) ABSTRACT

An exemplary vascular neural interface device/configuration and method can be provided for at least one of stimulating or recording the nervous system. For example, a package can be provided which can be inserted within a blood vessel. The package can include at least one transducer, at least one electrode, and at least one integrated circuit. The at least one transducer can receive or transmit a wireless signal which is used to provide energy or communicate with the at least one integrated circuit to at least one of record or stimulate the nervous system using recording electronics or stimulating electronics.

### CROSS-REFERENCE TO RELATED APPLICATION(S)

This application relates to and claims priority from U.S. Patent Application No. 63/189,536, filed on May 17, 2021, the entire disclosure of which is incorporated herein by reference.

### FIELD OF THE DISCLOSURE

The present disclosure relates generally to stimulating and recording techniques pertaining to the nervous system, and more specifically, to exemplary embodiments of exemplary system, device, method and computer-accessible medium which comprises and/or associated with a wireless stent-based interface for stimulating and recording a nervous system.

### BACKGROUND OF THE INVENTION

Electrical control of the nervous system has wide impact in neurology and other fields of medicine [see, e.g., Refs. 1-7]. However, the performance of electrodes implanted directly in the brain may degrade over time due to gliosis and other brain responses [see, e.g., Refs. 8 and 9], and strategies such as deep brain stimulation (DBS), despite widening application [see, e.g., Refs. 10-13], may require opening of the skull and invasive neural implantation.

Thus, it may be beneficial to provide an exemplary system, device, method and computer-accessible medium including a wireless stent-based interface for stimulating and recording a nervous system, which can overcome at least some of the deficiencies described herein above.

### SUMMARY OF EXEMPLARY EMBODIMENTS

An exemplary system, method and computer-accessible medium, according to exemplary embodiments of the present disclosure, can address, for example, these issues by developing vascular neural interfaces (VNIs), chronic, wirelessly-powered-and-controlled multifunctional devices that fit flush with vascular walls and that can be delivered in a minimally invasive fashion. An exemplary system, method and computer-accessible medium, according to exemplary embodiments of the present disclosure, can have electrodes that can span the circumference of the vessel when deployed, an application specific integrated circuit (ASIC), energy storage elements, and one or more ultrasound transducers for wireless powering and telemetry.

The exemplary system, method and computer-accessible medium, according to exemplary embodiments of the present disclosure, can facilitate a minimally invasive route to access virtually any organ. Every year nearly half-a-million [see, e.g., Ref. 14] catheterization procedures are performed in the US alone, including in the central nervous system (CNS) [see, e.g., Ref. 15]. Acute transvascular neural recording [see, e.g., Refs. 16-21] and stimulation of nerves [see, e.g., Ref. 22-24] with catheters has been described in both animal models and patients, and computational modeling has shown the feasibility of intravascular stimulation for DBS application for a variety of conditions [see, e.g., Refs. 25 and 26], suggesting the approach is sound. Recently, the first demonstration of a long-term chronic intravascular neural interface [see, e.g., Refs. 27 and 28] was described, offering further evidence of the value of this strategy. However, these approaches have employed wired devices, making them susceptible to breakage and other wiring-related complications. Such problems may account for a majority of the hardware-related failures in DBS (which in the literature can range in incidence from 1.8% to 48.5% of patients [see, e.g., Ref. 29]). The presence of long and possibly thin wires, traversing a curved vascular course, poses an even greater risk of wire breakage. Over the term of a chronic implant, the viability of long lengths of wire within the mobile vessel segments in the neck and, for arterial implants, within the highly mobile vessels in the region of the aortic arch is a widely recognized source of possible failure [see, e.g., Ref. 27]. In addition to mechanical fatigue, differential thrombogenicity and differential degrees of endothelialization over the length of the wire can be a source of problem with some segments of the wire opposed to the endothelium and with other segments within the lumen displaying different degrees of immobilization because of vessel curvature and geometry [See, e.g., Refs. 30 and 31]. All of these factors may present a chronic risk of thrombosis and ischemic infarct for implants placed on the arterial side and of venous stroke and hemorrhage for those on the venous side. This situation may be quite dissimilar to a fixed wireless implant that remains at its delivery site. Furthermore, such wiring would be expected to significantly disrupt blood flow even in medium-sized (2-3-mm) vessels, presenting a permanent risk of thrombosis once anticoagulation therapy [see, e.g., Ref. 27] is discontinued. Placement of wires in smaller vessels, in particular, can be extremely risky. These risks further limit translation of wired vascular nervous system interfaces to human use. Each of these technical challenges can grow with increases in the number of wires. The exemplary system, method and computer-accessible medium, according to exemplary embodiments of the present disclosure, can facilitate several devices to be simultaneously implanted in the same patient to produce interfaces across multiple brain and body regions.

By taking advantage, for example, of modern microfabrication and integrated circuit (IC) design techniques, the exemplary system, method and computer-accessible medium, according to exemplary embodiments of the present disclosure, can overcome the limitations of wired approaches with a fully wireless, ultrasound-based VNI. The exemplary VNIs can enable stimulation of both the central nervous systems (CNS) and the peripheral nervous system (PNS), most notably the vagus nerve, in a much less invasive manner than current clinical approaches. Vagus nerve stimulation (VNS) is an FDA-approved treatment for select neurological and psychiatric conditions including epilepsy, treatment-resistant depression, and cluster headache [see, e.g., Refs. 32-37]. There is also growing interest in using VNS to treat other conditions, such as heart failure, rheumatoid arthritis, inflammatory bowel disease, ischemic stroke, and traumatic brain injury [see, e.g., Refs. 38-48], many of which can be characterized by inflammation. Vagus nerve simulation could also be used as an approach to control drug-resistant hypertension [see, e.g., Ref. 49], which affects 40 million patients [see, e.g., Ref. 50]. Current vagus nerve stimulators require open surgical implantation which may be invasive and carries risks of injury to surrounding structures, general anesthesia, skin breakdown, exposed hardware, and infection [see, e.g., Ref. 51]. Additionally, these devices may be large. A conventional vagus nerve stimulator [see, e.g., Ref. 52] displaces a volume of more than 35,000 mm³, compared to only 1-2.5 mm³ for the exemplary VNI devices described herein. Transvascular vagal nerve stimulation can offer a less invasive and lower risk alternative to the surgically implanted stimulators currently in use.

The exemplary system, method and computer-accessible medium, according to exemplary embodiments of the present disclosure, also can act as a minimally invasive brain-machine-interface (BMI) device for accessing the CNS, thereby offering stable performance over time that can make brain-directed control of prosthetics a widespread option. By avoiding penetration into the brain parenchyma, for example, the exemplary VNIs can design around, the common gliosis- and scarring-induced signal degradation of implanted electrodes [see, e.g., Ref. 9]. The VNI platform can offer the potential for many recording channels through the implantation of an arbitrary number of devices. Deep brain stimulation (DBS) could also benefit from the use of VNIs [see, e.g., Ref. 25], because VNIs offer reduced invasiveness both pen- and post-procedure, with a minimal risk for the lesional effects present for at least some conventional DBS targets [see, e.g., Refs. 53 and 54]. Overall, VNIs can, for example, reduce patient risks, expand the therapeutic applications of neural stimulation and recording, and increase the number of patients who can potentially benefit [see, e.g., Refs. 55-59].

An exemplary system, method and computer-accessible medium, according to exemplary embodiments of the present disclosure, can offer a simple ultrasound-powered electrical stimulation device that can be delivered in a minimally invasive way from a distal access point (e.g., the femoral artery in rabbit when targeting the common carotid artery). The exemplary system, method and computer-accessible medium, according to exemplary embodiments of the present disclosure, can be wireless, stent-less devices, which include integrated circuits bonded to flexible substrates, whose intrinsic elasticity allow apposition with the vessel walls. The exemplary system, method and computer-accessible medium, according to exemplary embodiments of the present disclosure, was implemented in two different vascular-neural-interface devices (VNI1 and VNI2). VNI1 can deliver voltage or current stimulation pulses to a single electrode pair, while VNI2 can facilitate recording and stimulation for up to four channels at a 9.8-kHz bandwidth, enough to record action potentials (APs) in addition to local field potentials (LFPs). The ability to mechanically integrate the ASIC chips for both VNI1 and VNI2 into custom-fabricated, flexible polyimide (PI) packages has been demonstrated and in vivo deployment of the VNI1 design has been performed successfully. The resulting architecture can be, for example, modular with electrodes combined with the ASIC chip and piezoelectric transducers (to support powering and data transfer) in a single flexible package. Some have been exploring ultrasound for powering of and communication with implanted devices [see, e.g., Refs. 60-63]. What makes the exemplary embodiments of the present disclosure unique can be, for example, both the modular flexible packaging that maximizes volumetric efficiency (the amount of function within a given unit of displaced volume), the presence of techniques to make energy harvesting and communication independent of device orientation, and the introduction of “guide stars” into the package design which allow the implant to be located with an ultrasound imager in complex in vivo environments.

For example, using the vasculature as a route for electrical interaction with adjacent tissue may substantially depart from the current paradigms of clinical neuromodulation. VNIs can provide a minimally invasive alternative, compared to existing neural interfaces, which can significantly reduce patient burden, expand the therapeutic applications of neural stimulation and recording, and increase the number of patients that can potentially benefit. The exemplary system, method and computer-accessible medium, according to exemplary embodiments of the present disclosure, is the first fully wireless, chronic, transvascular platform for neural stimulation and recording. Although there is a long history of attempts to record neural activity [see, e.g., Refs. 16 and 64] and stimulate nerves [see, e.g., Refs. 22 and 24] from within blood vessels, these previous attempts (mostly based on conventional catheters) have been wired, i.e., not wireless, and present challenges for long-term implantation. Thus, exemplary embodiments of the present disclosure can be highly innovative and beneficial. For example, VNIs can be provided flush with the vessel walls, reducing the risk of thromboembolic and hemodynamic complications, and enhancing their ability to record and stimulate adjacent tissue [see, e.g., Ref. 25]. Another highly innovative aspect of exemplary embodiments of the present disclosure can be, for example, the use of ultraflexible electronics [see, e.g., Refs. 65-67], which can incorporate complementary metal-oxide-semiconductor (CMOS) devices directly at the site of the stent [see, e.g., Refs. 68 and 69]. Although flexible electronics have been developed before [see, e.g., Ref. 70], they may not be capable of the high performance required to support data conversion, telemetry, and wireless powering. The exemplary system, method and computer-accessible medium, according to exemplary embodiments of the present disclosure, can include the post-processing steps required to render conventional CMOS ICs completely flexible through aggressive thinning as well as the process required to fabricate stable biocompatible recording electrodes on flexible substrates which also connect to these ICs. When the die is milled to less than 12-μm, a bending stiffness of ˜5 μNm can result, which can be a six order-of-magnitude increase in mechanical compliance. Initial studies indicate that CMOS performance characteristics of thinned electronics remain comparable to that of the unthinned die [see, e.g., Refs. 71 and 72].

The exemplary system, method and computer-accessible medium, according to exemplary embodiments of the present disclosure, can provide a minimally invasive way to access the nervous system throughout the human body. Extensive clinical experience with stents suggests that such devices may safely integrate with living tissues, providing stable, long-term interfaces. Wirelessly powered and controlled stent-like electrical neural interface devices can have the potential to provide many new capabilities, for example, deep brain stimulators (DBS) that do not require the opening of the skull, improved brain-machine interfaces (BMIs) for control of prosthetics, and vascular vagus nerve stimulators for the management of treatment-resistant hypertension. This disclosure presents an exemplary vascular neural interface (VNI) technology, chronically implanted, wirelessly-powered-and-controlled device for neural stimulation and recording that can sit flush against blood vessel walls, without physically penetrating into the surrounding tissue, and that can be delivered in a minimally invasive fashion. These exemplary VNIs, can be based on self-expanding flexible microfabricated electrode arrays, and thinned, flexible complementary metal-oxide semiconductor (CMOS) electronics. The flexible CMOS chip, embedded in the wall of the stent, along with one or more piezoelectric transducers facilitate wireless, ultrasound-based power delivery, data communication, and control.

According to the exemplary embodiments of the present disclosure, a vascular neural interface device/configuration and method for at least one of stimulating or recording information of a nervous system can be provided. For example, a package (e.g., housing) can be provided which is configured to be inserted within a blood vessel. The package can include at least one transducer, at least one electrode, and at least one integrated circuit. The transducer(s) can be used to receive and/or transmit a wireless signal which is used to at least one of provide energy to or communicate with the at least one integrated circuit to at least one of record information of or stimulate the nervous system using recording electronics or stimulating electronics.

In an exemplary embodiment of the present disclosure, the transducer can be a piezoelectric transducer configured to interface with ultrasound energy. In addition or separately, the package can be a flexible circuit board. It is possible to have the package be deployed with a catheter into the blood vessel by, e.g., rolling the package around the catheter to form a rolled catheter configuration, and deploying the rolled catheter configuration at a predetermined location by expanding the catheter configuration against walls of the blood vessel. For example, the flexible circuit board can include polyimide and metal interconnects. In addition, the electrode(s) can span fully between opposing sides of the at least one flexible circuit board, such that when unrolled in the blood vessel, the electrode(s) and the flexible circuit board(s) can collectively span a circumference of the blood vessel. The integrated circuit(s) can have a configuration and dimensions to be mechanically flexible.

According to another or complementary exemplary embodiment of the present disclosure, the transducer(s) can be utilized to facilitate powering and communication with an external device that is rotationally invariant in the blood vessel. It is also possible to, using a data transmission arrangement, to transmit data to an external device using an amplitude shift keying procedure. Further, a data transmission arrangement can be utilized to transmit data from an external device using a load shift keying procedure and/or a modulated backscatter procedure.

The transducer(s) can be utilized to provide signals to locate an implant in an ultrasound imager using microbubbles which are provided into a cavity in the package.

In still another exemplary embodiment of the present disclosure, an external device can be provided outside of a body which is mounted on a surface of the vascular neural interface device/configuration at a particular location for powering and data transmission thereof. The external device can be an ultrasound transducer. The ultrasound transducer can be a two-dimension array of transducers. The two-dimensional array of transducers can be a wearable patch device.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1A is a side perspective view of an exemplary VNI device according to an exemplary embodiments of the present disclosure;

FIG. 1B is a side perspective view of a blood vessel with the exemplary VNI device of FIG. 1A placed therein;

FIG. 2A is a top view of an illustration of an exemplary packaged VNI1 according to an exemplary embodiment of the present disclosure prior to implantation;

FIG. 2B is an illustration of the exemplary VNI1 device(s) according to an exemplary embodiment of the present disclosure rolled and inserted into an exemplary microcatheter delivery system;

FIG. 2C is an exemplary fluoroscope image of the exemplary inserted VNI1 device according to an exemplary embodiment of the present disclosure;

FIG. 3A is an exemplary BMode image of an exemplary acoustic guidestar system on the exemplary flexible package according to an exemplary embodiment of the present disclosure;

FIG. 3b is a graph of an exemplary frequency spectrum fingerprint of the acoustic guidestar response with imaging at 2.5 MHz and a response at 5 MHz using the system, package and device according to exemplary embodiment of the present disclosure;

FIG. 4A is an illustration of an exemplary blood pressure recording, with lighter lines indicating the periods of stimulation, according to the exemplary embodiments of the present disclosure;

FIG. 4B is an illustration of an exemplary diastolic pressure for each cardiac cycle, in black during baseline periods, and in lighter shade during stimulation, according to the exemplary embodiments of the present disclosure;

FIG. 5 is an block diagram of the exemplary VNI2 system according to an exemplary embodiments of the present disclosure; and

FIG. 6 is a side view illustration of an exemplary link between an exemplary external acoustic device, through soft tissue to the implanted VNI device, according to the exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

Exemplary system, method and computer-accessible medium, according to exemplary embodiments of the present disclosure, can include and/or provide one or more self-expanding flexible devices which allow deployment in tortuous vessels and enhance the devices' flexibility while rolled or folded during delivery.

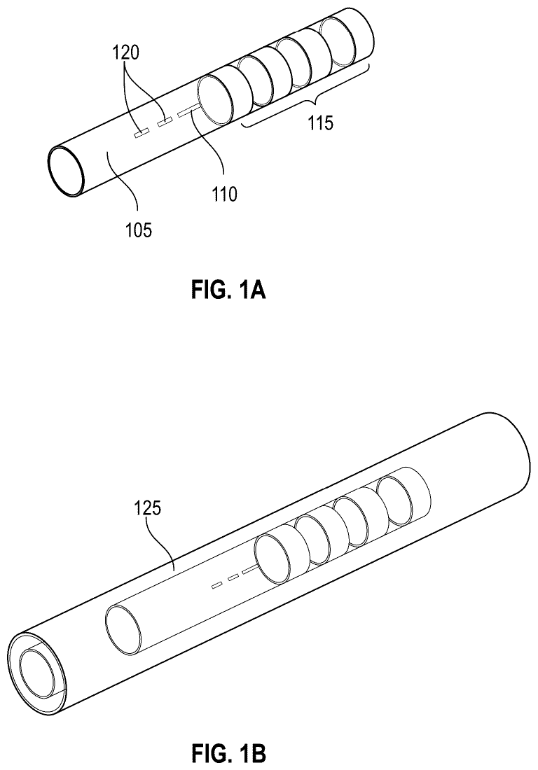

For example, FIG. 1A shows a side perspective view of an exemplary VNI device according to an exemplary embodiments of the present disclosure. The exemplary VNI device illustrated in FIG. 1A can be configured for—at least—an electrical stimulation. These exemplary devices can include custom 7-μm polyimide substrates 105 with patterned electrode arrays 115, and bond the existing ASICs 110 and piezoelectric transducers 120 to produce both VNI1 and VNI2 devices. In one non-limiting example, such exemplary VNI devices can include, e.g., 1.5 mm-wide gold electrodes (gold colored), 350 μm-wide ASIC (light-gray), 350 μm by 770 μm PMN-PT piezoelectric transducers (dark gray), 10-μm-thick polyimide substrate (transparent). The substrate would not necessarily be a single continuous sheet. FIG. 1B shows a side perspective view of a blood vessel 125 with the exemplary VNI device illustrated FIG. 1A placed therein.

The exemplary wireless VNI device can utilize ultrasound (US) signals for power and communication, giving the devices, for example, two distinct advantages. For example, the acoustic phase velocity of the US waves through soft tissue (˜1540 m/s) can support wavelength-determined device sizes at the submillimeter scale for MHz frequencies [see, e.g., Ref. 73]. Second, the low attenuation of US in soft tissue, on average ˜0.7 dB/cm/MHz, can allow powering the devices at depths of up to 5 cm at 2 MHz. In contrast, the attenuation of electromagnetic energy may be considerably more severe at 14.6 dB/cm at 3 GHz [see, e.g., Ref. 61].

The use of conventional metal stents may introduce problems for US data telemetry due to reflections from the stent itself. The exemplary VNIs do not need to maintain a stenotic artery pattern (as traditional stents), and instead simply should hold electrodes against the vessel wall. As such, the exemplary VNIs according to exemplary embodiments of the present disclosure can utilize the elasticity of the flexible substrate (e.g. polyimide, which is highly compatible with endothelial cells [see, e.g., Ref. 74] and has good hemocompatibility [see, e.g., Ref. 75]) itself to deploy and maintain or hold the electrodes and active electronics in place until it is fully integrated into the tissue. For particularly flexible substrates, van der Waals forces can also contribute to the adhesion to the vessel wall, further ensuring tight apposition. This exemplary approach according to exemplary embodiments of the present disclosure, among others, can eliminate interference with US-based powering and communication, as well as facilitate extremely thin VNIs, with thickness on the order of a few micrometers, which can greatly reduce hemodynamic complications and facilitate integration into the tissue (hence greatly reducing the risk of thromboembolic and restenotic complications). Single-micron thick packaging is also critical to enabling the safe deployment of VNIs in small, i.e. <about 1 mm, vessels than what is viable with traditional/nitinol stent-based devices.

An extensive system for in vitro testing was set up to assess the ability of the VNI delivery system (i.e. the VNI itself plus the catheter in which it is mounted) to navigate sets of (e.g. 1-3) turns of specified inner radii (e.g. 2 to 20 mm) within mock silicone vessels, while also recording the force necessary to cause the device to do so. A 3D-printed model of the relevant vasculature was used as a testbed. Furthermore, the ability of the VNI to be successfully deployed under high-flow conditions (e.g. 50% more than the maximum expected in vivo) was assessed. The performance of the exemplary device was compared to that of a conventional neurovascular stent delivery system (e.g., Wingspan®, Boston Scientific). The ability to tightly abut the vessel walls is also a key parameter assessed in these designs. The use of silicone mock vessels can facilitate the high magnification observation of the deployed devices. A colored dye can also be injected through the mock-vessel, to help reveal if any part of the device is not fully conformal to the vessel wall.

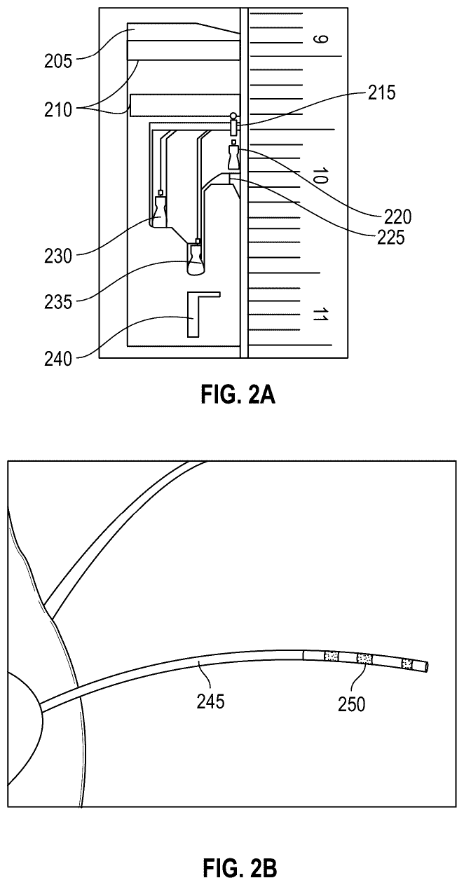

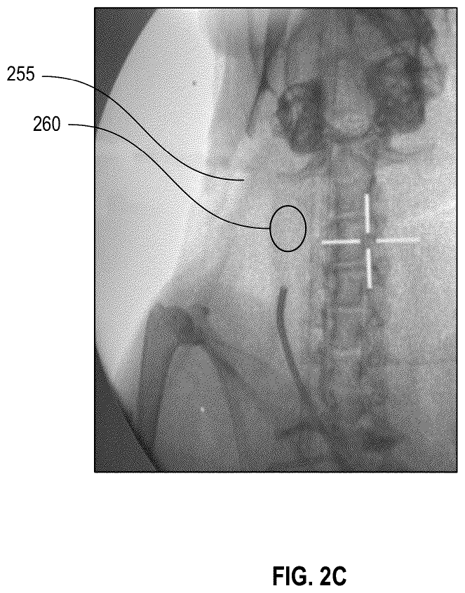

FIG. 2A shows a top view of an illustration of an exemplary flexibly-packaged VNI1 according to an exemplary embodiment of the present disclosure prior to implantation. For example, in VNI1, biphasic current pulses can be transmitted to electrodes 210 on the flexible package 205, as illustrated in FIG. 2A. VNI1 can include, e.g., an ultrasound link to receive power (by rectifying the transduced acoustic energy) and/or data (for commands and configuration) on an amplitude-shift-keying (ASK) modulated 2-MHz carrier frequency. This exemplary link can rely on one or more (e.g., three) external lead magnesium niobate, lead titanate (PMN-PT) piezoelectric transducers 220, 230, and 235, (e.g., which can be fully encapsulated to avoid cytotoxicity) mounted on the flexible package 205. It may be difficult to control the radial orientation of the VNI during implantation. Since the power transfer to the implanted VNI can be a function of the angle of the incident ultrasound to the implanted transducer, it is possible that insufficient power may be transmitted in the case that the implanted device is perpendicular to the external interface. The use of, for example, transducers 220, 230, and 235 (and possibly a number of—e.g. three—rectifiers), deployed around the circumference of the vessel can facilitate angle-insensitivity of powering and communication after delivery. Most or all of the transducers can be spatially offset along the length of the VNI to minimize the risk of vessel occlusion.

VNI1 may utilize, for example, 4 msec to generate its stimulation supply from reset and may buffer that supply using an external energy storage element 225. Thus, VNI1 can deliver stimulation pulses at a maximum rate of, e.g., about 200 Hz (e.g., about 4 msec for supply generation, about 1 ms for pulse delivery and charge redistribution; twice the rate most applications require). The stimulation pulse repetition frequency can be defined by the delivery of acoustic pulses from the external probe facilitating flexibility that can be tuned to the specific demands of the stimulation target and in vivo application. VNI1 can deliver biphasic constant-current pulses of up to 1 mA on steps of 15 μA, and can drive electrodes of arbitrary impedance, limited by the voltage compliance of the stimulation supply. The conversion of acoustic energy to electrical stimulation pulses and the development of the external voltage supply can be facilitated by CMOS IC 215.

As shown in FIG. 2A, in an exemplary non-limiting exemplary embodiment, the exemplary flexible packaging 205 can be fabricated by laser micromachining polyimide (PI) sheets which can be, e.g., approximately 7-μm thick using an excimer micromachining tool. A single layer of Ti/Au interconnects can be fabricated using standard photolithographic techniques. For example, the exemplary package can also contain, e.g., two 1.5-mm-wide electrodes 210 spanning the circumference of the vessel (analogous to DBS ring electrodes) with an electrode impedance of approximately 10 kΩ (values comparable to commercial DBS electrodes [see, e.g., Refs. 76 and 77]). Ti/Au pads on the package can match the pad positions on the integrated circuit; lithographically defined 1-μm-thick copper pillars provide the via metal from the package to the pads of the ASIC and an 8-μm anisotropic conducting film provides the adhesive underfill and conductive interface to hold the chip in place. The Finetech Fineplacer Lambda tool can be used to flip-chip position the ASIC 215 pads to the package and perform the bonding by heating to 180° C. The 350-μm-thick PMN-PT transducers, cut to dimensions of 350 μm by 770 μm (oriented along the length of the vessel wall in-line with the IC) with a DISCO dicing saw, are mounted to the package with adhesive thin layer of H20E low temperature conductive epoxy. A thin layer of polydimethylsiloxane (PDMS) is used to encapsulate and passivate the transducer and the chip.

The exemplary flexible packaging configuration according to the exemplary embodiments of the present disclosure can reduce or even eliminate interference from the hyperechogenic wire mesh stent used in conventional stenting procedures, and the optimal power and data transfer to the implant is traded in exchange for complexity in finding the implant under low-frequency acoustic guidance. For example, in B-Mode images, the tiny VNI1 can be approximately one wavelength long (when thinned to −10 μm the integrated circuit chip itself becomes acoustically transparent at 2 MHz), and the other implanted materials may not be robust acoustic reflectors. As a result, finding the implanted device with the external probe may become challenging. In acute studies, the guide catheter provides a means of placing a second wired transducer to ping back to the ultrasound probe near the implanted device. The exemplary guidewire and guide catheter system can also be distinguishable using B-Mode imaging; however, these interfaces may only be available during surgical insertion of the device. To achieve chronic discovery of the exemplary implant, in an exemplary embodiment of the present disclosure, an acoustic “guide star” 240 can be utilized that is, e.g., 0.5 μL of acoustic contrast agent (Lantheus DEFINITY microbubbles, 3-10 μm in diameter) sealed in a microfluidic cavity 5-10 acoustic wavelengths long. While others have suggested using the acoustic transducer in a beacon mode or looking for the third harmonic of an active acoustic front end, this is may be, but not necessarily, impractical in the exemplary application(s) of the present disclosure [see, e.g., Ref. 78] for two reasons. For example, the third harmonic generated from the full wave rectifier square pulses may be reflected at about 6 MHz, requiring a wide bandwidth transducer for low MHz acoustic waves, well beyond the capabilities of the exemplary ATL P4-1 probe. Additionally, for situations in which the transducer is implanted in a misaligned orientation with respect to the external probe, no beacon signal may be detectable from the implant as misalignment attenuates delivered power as a function of the cosine of the offset angle [see, e.g., Ref. 63]. The acoustic contrast agent may be an isotropic reflector of acoustic energy at even harmonics of excitation, limiting the bandwidth requirements for the external probe and ensuring detection regardless of implantation angle.

To preferentially image the acoustic guide star, the exemplary system according to an exemplary embodiment of the present disclosure can utilize a procedure termed “pulse inverse imaging.” In this imaging modality, e.g., two imaging pulses can be used per ray line in rapid succession (120 μs apart), the first with a positive excitation direction and the second with a negative excitation direction. The resulting pulse-echo responses can be summed. As a result, linear responders, including biological tissues, can be significantly reduced or eliminated leaving only nonlinear responders in the image 320. For example, an infinite impulse response filter around the second harmonic can be used to further remove background, leaving a dark field except for the acoustic guide star.

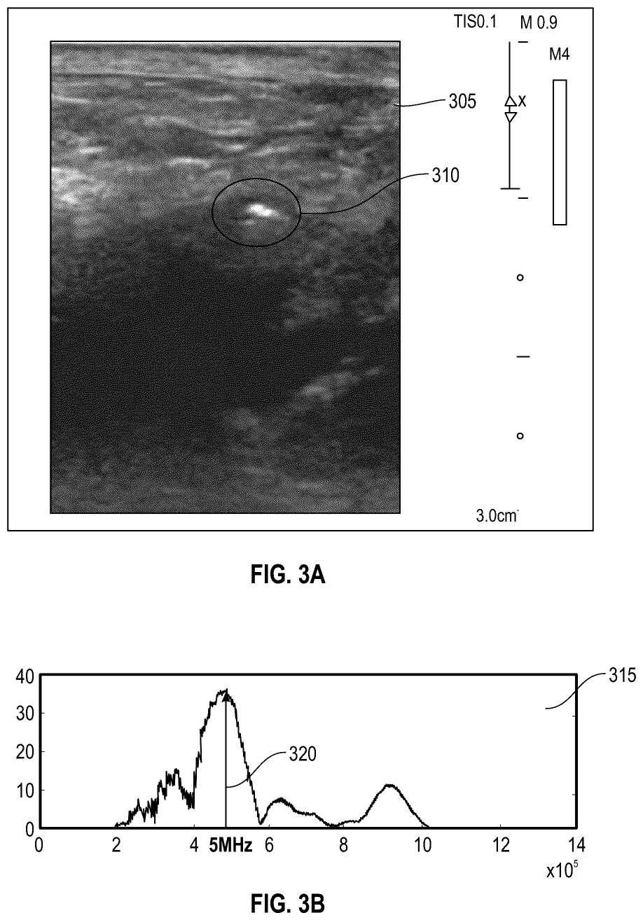

FIG. 3A shows an exemplary BMode image of an exemplary acoustic guidestar system 310 on the exemplary flexible package according to an exemplary embodiment of the present disclosure, FIG. 3B ill a graph of an exemplary frequency spectrum fingerprint of the acoustic guidestar response 315 with imaging at 2.5 MHz and a response at 5 MHz using the system, package and device according to exemplary embodiment of the present disclosure. To increase or even maximize power delivery, the exemplary device according to the exemplary embodiments of the present disclosure can take B-mode 305 and PII ultrasound images [see, e.g., Ref. 79], and analyze the frequency response 315 of the exemplary PII image using the Verasonics Vantage system. The precise location of the implanted device 310 can be determined using guide stars and direct ultrasound to the piezoelectric transducer with phased wavefronts focused on the implanted transducer element. The incident ultrasound carrier amplitude envelope can be modulated to encode data to control the implanted device. Recording data can be transmitted by means of energy backscattering, in which ultrasound pressure waves can be absorbed or reflected at a secondary transducer representing binary ‘1’s or ‘0’s. To detect these backscattered ultrasound waves, the exemplary device according to exemplary embodiment of the present disclosure can be configured, e.g., about 10% of the Verasonics System external transducer array to continuously image from the implant and parse the received data in Matlab.

FIG. 2B shows an illustration of the exemplary VNI1 device(s) 250 according to an exemplary embodiment of the present disclosure rolled and inserted into an exemplary microcatheter delivery system. The exemplary device(s) 250 according to the exemplary embodiments of the present disclosure can be loaded into a 4 Fr (1.3 mm OD, comparable to an 18 Ga needle) delivery system 245, based on commercial microcatheters. FIG. 2C illustrates an exemplary fluoroscope image of the exemplary inserted VNI1 device according to an exemplary embodiment of the present disclosure which provides a validation of the efficacy of the self-deployment strategy in vivo, with both dummies (n=9 devices, n=5 rabbits) and actual VNI1 devices (n=3 devices, n=3 rabbits), deployed in the common carotid artery 260 of the rabbit 255 (which has a diameter on the order of 1.5-2 mm, comparable to human cortical veins).

The exemplary surgical procedure according to an exemplary embodiment of the present disclosure can be compatible with the minimally invasive stenting procedure which more than 2 million people receive each year. First, in the exemplary procedure according to the exemplary embodiment of the present disclosure, a distal vascular access point is opened, for example the femoral artery, by blunt dissection and a catheter introducer can be placed. For example, the surgeon then navigates a 5 Fr guide catheter under fluoroscopic guidance from the femoral access point to the desired deployment target, for example the common carotid artery, slightly caudal to the carotid bifurcation. The guidewire can be removed and the delivery vehicle containing the device can be placed at the delivery site. As the delivery vehicle is retracted, the device can self-expand to the vessel extents, holding the VNI in place and ensuring the electrodes remain in tight apposition to the vessel walls. In all cases, the vessels 260 remained patent, as assessed under fluoroscopy via contrast agent injection, up to the longest duration tested, which was three hours, as shown in the illustration of FIG. 2B.

The delivery vehicle can be removed and the Verasonics ultrasound system with the ATL P4-1 probe can be placed on the animal's skin with acoustic coupling gel. Device location is then determined using a harmonic imaging technique and analysis of the frequency response of the acoustic response 315 (as shown in FIG. 3B), which cancels the linear soft-tissue in the imaging plane, leaving only the nonlinear guide star response, which exhibits a strong second harmonic peak 320 in the frequency spectrum. Upon determining the device location, e.g., 7-ms ultrasound pulses at a 200-Hz repetition rate can be delivered. Each 7-ms acoustic pulse can be sufficient to power up the implanted VNI, allow its stimulation voltage rail to stabilize, and deliver a 300 μs-long biphasic current pulse of 300 μA at each ultrasound pulse repetition.

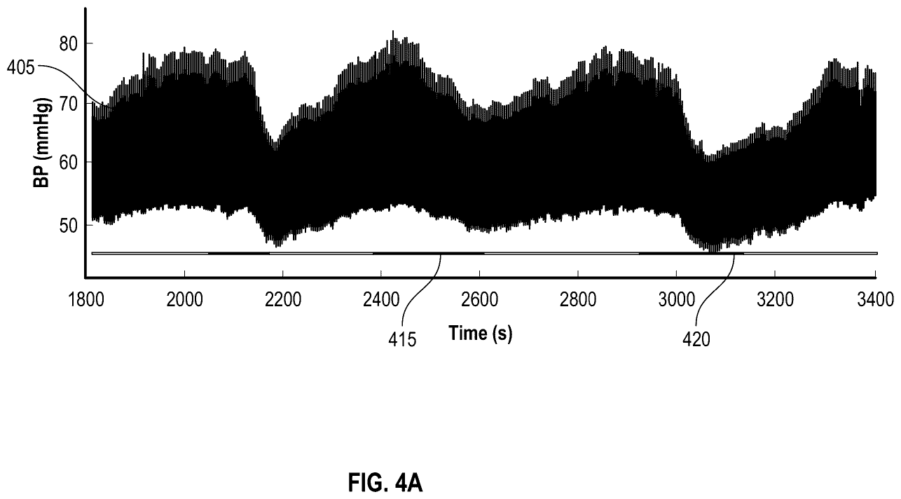

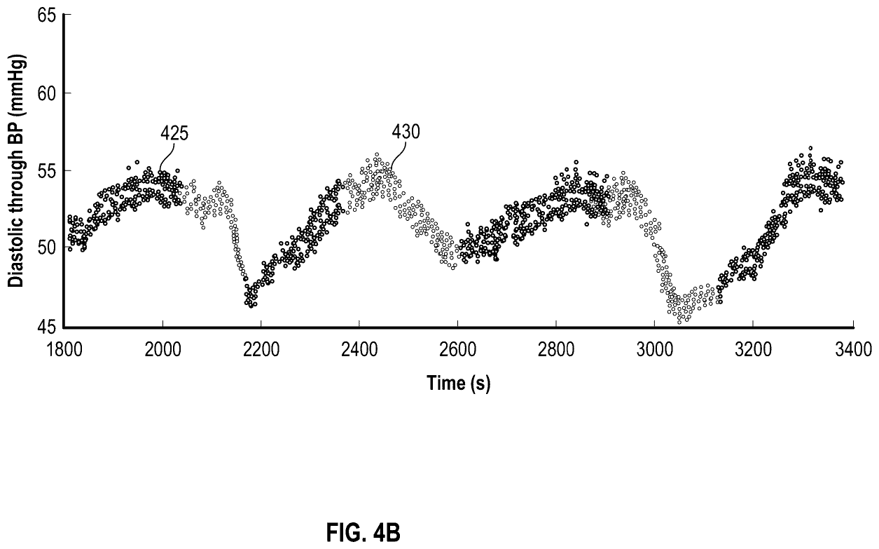

FIG. 4A provides an illustration of an exemplary blood pressure recording 405, with lighter lines indicating the periods of stimulation, according to the exemplary embodiments of the present disclosure. FIG. 4B shows an illustration of an exemplary diastolic pressure 425 for each cardiac cycle, in black during baseline periods, and in lighter shade 430 during the stimulation, according to the exemplary embodiments of the present disclosure. For example, as shown in FIGS. 4A and 4B, the blood pressure of the animal 405, 425 was recorded over the duration of the in vivo experiment and monitored prior to stimulation, and during the stimulation epochs 410, 415, 420. These stimulation parameters effectively elicited the expected physiological response, a reduction in blood pressure, when the devices were powered. Importantly, no physiological effect was elicited by the focused ultrasound pulse train alone, when it was focused away from the piezoelectric transducer on the vessel itself.

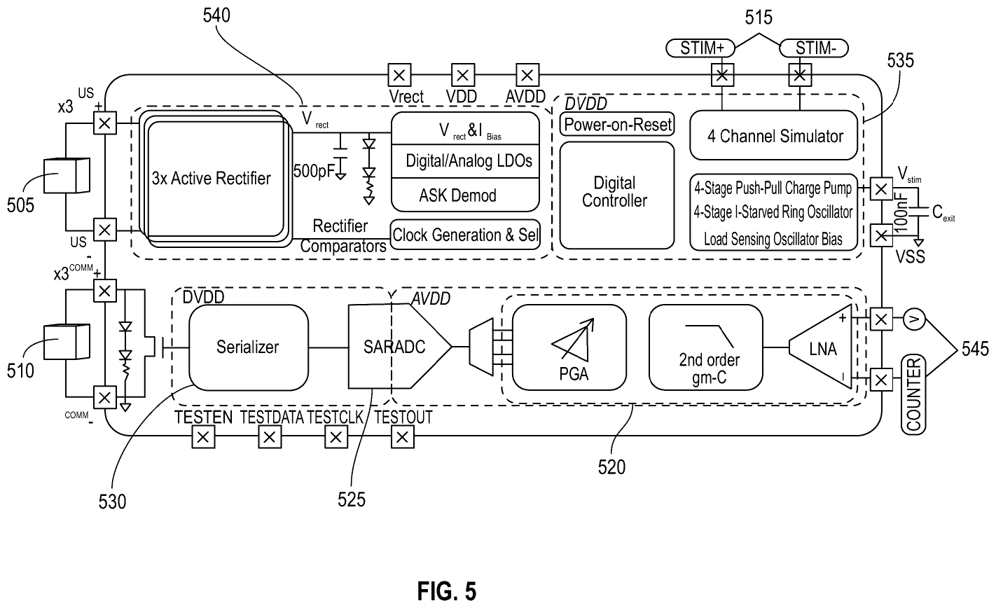

FIG. 5 shows an block diagram of an exemplary VNI2 system according to an exemplary embodiments of the present disclosure For example, VNI2 can be similarly powered by a plurality (e.g., three) spatially offset piezoelectric transducers 505, and can include the same or similar power conditioning circuits 540 as VNI1. As another example, VNI2 can select between a plurality (e.g., four total) stimulation channels 515, and deliver biphasic constant-current pulses of up to about 1 mA on steps of about 15 μA, which can be limited by a the stimulation supply. The exemplary neural recording system according to the exemplary embodiment of the present disclosure can connect to stimulation electrodes 515 or a separate set of recording electrodes 545, and can include a low noise fully differential amplifier chain 520 with a programmable mid-band gain of 39-60 dB, low frequency roll off at 5.9 Hz and high frequency roll off at 9.8 kHz. The amplifier noise of the exemplary LNA system is 6.48 μV/√ (Hz) between 5.9 Hz and 9.8 kHz. The recording chain can drive a 10-bit-resolution split-capacitor successive-approximation-register (SAR) ADC 525. The resulting digitized data can be transmitted serially using, e.g., a serializer 530 with load-shift-keying of a second set of piezoelectric transducers 510, modulating the backscatter of the same 2-MHz ultrasound pressure waves at a data rate of 72 kbps, limited by the channel capacity of the LSK link such that the VNI2 data stream can be reliably reconstructed from the measured ultrasound backscatter.

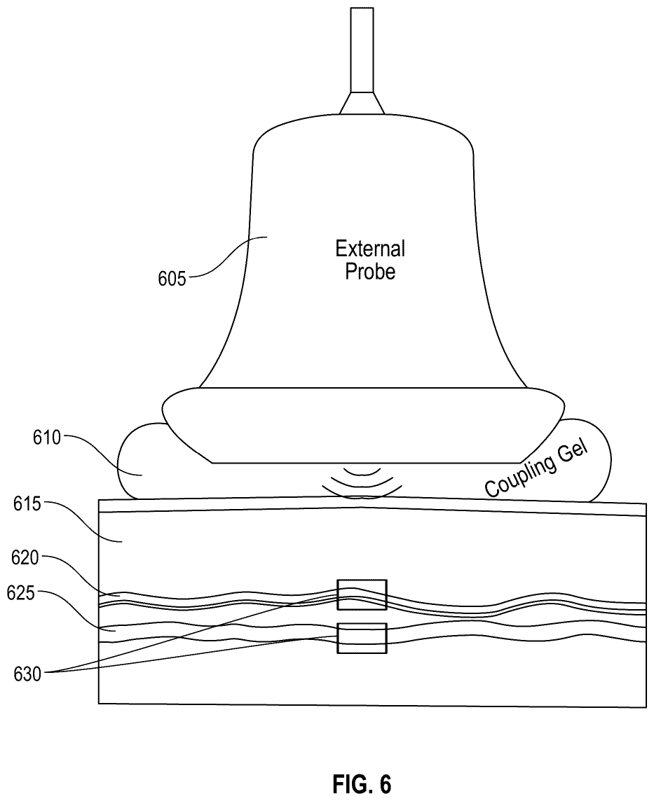

FIG. 6 provides a side view illustration of an exemplary link between an exemplary external acoustic device or probe 605 and an implanted VNI 630, through soft tissue (which may reside in a vein 625 or an artery 620) to the implanted VNI device 630, according to the exemplary embodiments of the present disclosure. As shown in FIG. 6, the exemplary link between the external device 605 and the exemplary implanted VNI 630 can be provided through an acoustic coupling gel 610 and soft tissue 615. The outside-the-body device or probe 605 interfaced to the exemplary VNI devices can be or include, for example, a linear ultrasound probe, a focused single transducer and/or a two-dimensional phased array. In some exemplary embodiments of the present disclosure, the two-dimensional phased array can be used because it can be focused on the VNI without the need for the two-dimensional phased array to be mechanically moved. In addition, these exemplary two-dimensional arrays can be fabricated in a wearable, patch form factor.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following reference is hereby incorporated by references in their entireties:
1. Andersson U, Tracey K J. Neural reflexes in inflammation and immunity. The Journal of Experimental Medicine. 2012; 209(6):1057-68. doi: 10.1084/jem.20120571.
2. Birmingham K, Gradinaru V, Anikeeva P, Grill W M, Pikov V, McLaughlin B, Pasricha P, Weber D, Ludwig K, Famm K. Bioelectronic medicines: a research roadmap. Nat Rev Drug Discov. 2014; 13(6):399-400. doi: 10.1038/nrd4351.
3. Borovikova L V, Ivanova S, Zhang M, Yang H, Botchkina G I, Watkins L R, Wang H, Abumrad N, Eaton J W, Tracey K J. Vagus nerve stimulation attenuates the systemic inflammatory response to endotoxin. Nature. 2000; 405(6785):458-62. doi: 10.1038/35013070.
4. Famm K, Litt B, Tracey K J, Boyden E S, Slaoui M. Drug discovery: A jump-start for electroceuticals. Nature. 2013; 496(7444):159-61. doi: 10.1038/496159a.
5. Li M, Zheng C, Sato T, Kawada T, Sugimachi M, Sunagawa K. Vagal nerve stimulation markedly improves long-term survival after chronic heart failure in rats. Circulation. 2004; 109(1):120-4. doi: 10.1161/01.CIR.0000105721.71640.D A.
6. Hochberg L R, Serruya M D, Friehs G M, Mukand J A, Saleh M, Caplan A H, Branner A, Chen D, Penn R D, Donoghue J P. Neuronal ensemble control of prosthetic devices by a human with tetraplegia. Nature. 2006; 442(7099):164-71. doi: http://www.nature.com/nature/journal/v442/n7099/suppinfo/nature04970_S1.html.
7. Bouton C E, Shaikhouni A, Annetta N V, Bockbrader M A, Friedenberg D A, Nielson D M, Sharma G, Sederberg P B, Glenn B C, Mysiw W J, Morgan A G, Deogaonkar M, Rezai A R. Restoring cortical control of functional movement in a human with quadriplegia. Nature. 2016; 533(7602):247-50. doi: 10.1038/nature17435.
8. Liu X, McCreery D B, Carter R R, Bullara L A, Yuen T G H, Agnew W F. Stability of the interface between neural tissue and chronically implanted intracortical microelectrodes. IEEE Transactions on Rehabilitation Engineering. 1999; 7(3):315-26. doi: 10.1109/86.788468.
9. Polikov V S, Tresco P A, Reichert W M. Response of brain tissue to chronically implanted neural electrodes. Journal of Neuroscience Methods. 2005; 148(1):1-18. doi: http://dx.doi.org/10.1016/j.jneumeth/2005/08.015.
10. Anderson W S, Lenz F A. Surgery Insight: deep brain stimulation for movement disorders. Nat Clin Pract Neuro. 2006; 2(6):310-20. doi: 10.1038/ncpneuro0193.
11. Deuschl G, Schade-Brittinger C, Krack P, Volkmann J, Schafer H, Bötzel K, Daniels C, Deutschländer A, Dillmann U, Eisner W, Gruber D, Hamel W, Herzog J, Hilker R, Klebe S, Kloss M, Koy J, Krause M, Kupsch A, Lorenz D, Lorenzl S, Mehdorn H M, Moringlane J R, Oertel W, Pinsker M O, Reichmann H, Reuss A, Schneider G-H, Schnitzler A, Steude U, Sturm V, Timmermann L, Tronnier V, Trottenberg T, Wojtecki L, Wolf E, Poewe W, Voges J, German Parkinson Study Group N S. A randomized trial of deep-brain stimulation for Parkinson's disease. The New England Journal of Medicine. 2006; 355(9):896-908. doi: 10.1056/NEJMoa060281.
12. Greenberg B D, Malone D A, Friehs G M, Rezai A R, Kubu C S, Malloy P F, Salloway S P, Okun M S, Goodman W K, Rasmussen S A. Three-year outcomes in deep brain stimulation for highly resistant obsessive-compulsive disorder. Neuropsychopharmacology. 2006; 31(11):2384-93. doi: 10.1038/sj.npp.1301165.
13. Mayberg H S, Lozano A M, Voon V, McNeely H E, Seminowicz D, Hamani C, Schwalb J M, Kennedy S H. Deep Brain Stimulation for Treatment-Resistant Depression. Neuron. 2005; 45(5):651-60. doi: 10.1016/j.neuron.2005.02.014.
14. Riley R F, Don C W, Powell W, Maynard C, Dean L S. Trends in coronary revascularization in the United States from 2001 to 2009: recent declines in percutaneous coronary intervention volumes. Circ Cardiovasc Qual Outcomes. 2011; 4(2):193-7. doi: 10.1161/CIRCOUTCOMES.110.958744.
15. Grigoryan M, Chaudhry S A, Hassan A E, Sun F K, Qureshi Al. Neurointerventional procedural volume per hospital in United States: implications for comprehensive stroke center designation. Stroke; a Journal of Cerebral Circulation. 2012; 43(5):1309-14. doi: 10.1161/STROKEAHA.111.636076.
16. Bower M R, Stead M, Van Gompel J J, Bower R S, Sulc V, Asirvatham S J, Worrell G A. Intravenous recording of intracranial, broadband EEG. Journal of Neuroscience Methods. 2013; 214(1):21-6. doi: 10.1016/j.jneumeth.2012.12.027.
17. He B D, Ebrahimi M, Palafox L, Srinivasan L. Signal quality of endovascular electroencephalography. Journal of Neural Engineering. 2016; 13(1):016016. doi: 10.1088/1741-2560/13/1/016016.
18. Garcia-Asensio S, Guelbenzu S, Barrena R, Valero P. Technical Aspects of Intra-Arterial Electroencephalogram Recording. Interv Neuroradiol. 1999; 5(4):289-300. doi: 10.1177/159101999900500405.
19. Nakase H, Ohnishi H, Touho H, Karasawa J, Yamamoto S, Shimizu K. An intra-arterial electrode for intracranial electro-encephalogram recordings. Acta Neurochir. 1995; 136(1-2):103-5.
20. Zeitlhofer J, Feldner G, Heimberger K, Mayr N, Samec P. Transarterial EEG in superselective cerebral angiography. EEG EMG Z Elektroenzephalogr Elektromyogr Verwandte Geb. 1990; 21(1):70-2.
21. Watanabe H, Takahashi H, Nakao M, Walton K, Llinás R R. Intravascular neural interface with nanowire electrode. Electron Comm Jpn. 2009; 92(7):29-37. doi: 10.1002/ecj.10058.
22. Schauerte P N, Scherlag B J, Scherlag M A, Goli S, Jackman W, Lazzara R. Transvenous Parasympathetic Cardiac Nerve Stimulation. Journal of Cardiovascular Electrophysiology. 1999; 10(11):1517-24. doi: 10.1111/j.1540-8167.1999.tb00210.x.
23. Thompson G W, Levett J M, Miller S M, Hill M R, Meffert W G, Kolata R J, Clem M F, Murphy D A, Armour J A. Bradycardia induced by intravascular versus direct stimulation of the vagus nerve. Ann Thorac Surg. 1998; 65(3):637-42.
24. Ponikowski P, Javaheri S, Michalkiewicz D, Bart B A, Czarnecka D, Jastrzebski M, Kusiak A, Augostini R, Jagielski D, Witkowski T, Khayat R N, Oldenburg O, Gutleben K-J, Bitter T, Karim R, Iber C, Hasan A, Hibler K, Germany R, Abraham W T. Transvenous phrenic nerve stimulation for the treatment of central sleep apnoea in heart failure. Eur Heart J. 2012; 33(7):889-94. doi: 10.1093/eurheartj/ehr298.
25. Teplitzky B A, Connolly A T, Bajwa J A, Johnson M D. Computational modeling of an endovascular approach to deep brain stimulation. Journal of Neural Engineering. 2014; 11(2):026011.
26. Opie N L, John S E, Rind G S, Ronayne S M, Wong Y T, Gerboni G, Yoo P E, Lovell T J H, Scordas T C M, Wilson S L, Dornom A, Vale T, O'Brien T J, Grayden D B, May C N, Oxley T J. Focal stimulation of the sheep motor cortex with a chronically implanted minimally invasive electrode array mounted on an endovascular stent. Nature Biomedical Engineering. 2018; 2(12):907-14. doi: 10.1038/s41551-018-0321-z.
27. Oxley T J, Opie N L, John S E, Rind G S, Ronayne S M, Wheeler T L, Judy J W, McDonald A J, Dornom A, Lovell T J H, Steward C, Garrett D J, Moffat B A, Lui E H, Yassi N, Campbell B C V, Wong Y T, Fox K E, Nurse E S, Bennett I E, Bauquier S H, Liyanage K A, van der Nagel N R, Perucca P, Ahnood A, Gill K P, Yan B, Churilov L, French C R, Desmond P M, Home M K, Kiers L, Prawer S, Davis S M, Burkitt A N, Mitchell P J, Grayden D B, May C N, O'Brien T J. Minimally invasive endovascular stent-electrode array for high-fidelity, chronic recordings of cortical neural activity. Nature Biotechnol. 2016; 34(3):320-7. doi: 10.1038/nbt.3428.
28. Oxley T J, Yoo P E, Rind G S, Ronayne S M, Lee C S, Bird C, Hampshire V, Sharma R P, Morokoff A, Williams D L. Motor neuroprosthesis implanted with neurointerventional surgery improves capacity for activities of daily living tasks in severe paralysis: first in-human experience. Journal of neurointerventional surgery. 2021; 13(2):102-8.
29. Benabid A L, Chabardes S, Mitrofanis J, Pollak P. Deep brain stimulation of the subthalamic nucleus for the treatment of Parkinson's disease. The Lancet Neurology. 2009; 8(1):67-81. doi: 10.1016/S1474-4422(08)70291-6.
30. Foin N, Gutiérrez-Chico J L, Nakatani S, Toni R, Bourantas C V, Sen S, Nijjer S, Petraco R, Kousera C, Ghione M, Onuma Y, Garcia-Garcia H M, Francis D P, Wong P, Mario C D, Davies J E, Serruys P W. Incomplete Stent Apposition Causes High Shear Flow Disturbances and Delay in Neointimal Coverage as a Function of Strut to Wall Detachment Distance. Circulation: Cardiovascular Interventions. 2014; 7(2):180-9. doi: 10.1161/CIRCINTERVENTIONS.113.000931.
31. Rouchaud A, Ramana C, Brinjikji W, Ding Y H, Dai D, Gunderson T, Cebral J, Kallmes D F, Kadirvel R. Wall Apposition Is a Key Factor for Aneurysm Occlusion after Flow Diversion: A Histologic Evaluation in 41 Rabbits. American Journal of Neuroradiology. 2016; 37(11):2087-91. doi: 10.3174/ajnr.A4848.
32. Heck C, Helmers S L, DeGiorgio C M. Vagus nerve stimulation therapy, epilepsy, and device parameters: scientific basis and recommendations for use. Neurology. 2002; 59(6 suppl 4):S31-S7.
33. Ruffoli R, Giorgi F S, Pizzanelli C, Murri L, Paparelli A, Fornai F. The chemical neuroanatomy of vagus nerve stimulation. Journal of chemical neuroanatomy. 2011; 42(4):288-96.
34. Conway C R, Gott B M, Azhar N H. Vagus nerve stimulation for treatment-refractory depression. Neuromodulation in Psychiatry. 2016:335-52.
35. Láinez M J, Guillamón E. Cluster headache and other TACs: pathophysiology and neurostimulation options. Headache: The Journal of Head and Face Pain. 2017; 57(2):327-35.
36. Kumar A, Bunker M T, Aaronson S T, Conway C R, Rothschild A J, Mordenti G, Rush A J. Durability of symptomatic responses obtained with adjunctive vagus nerve stimulation in treatment-resistant depression. Neuropsychiatric disease and treatment. 2019; 15:457.
37. Pisapia J, Baltuch G. Vagus nerve stimulation: Introduction and technical aspects. Neuromodulation in Psychiatry. 2015:325-34.
38. Zhang Y, Popovic Z B, Bibevski S, Fakhry I, Sica D A, Van Wagoner D R, Mazgalev T N. Chronic vagus nerve stimulation improves autonomic control and attenuates systemic inflammation and heart failure progression in a canine high-rate pacing model. Circulation: Heart Failure. 2009; 2(6):692-9.
39. Bonaz B, Picq C, Sinniger V, Mayol J-F, Clarengon D. Vagus nerve stimulation: from epilepsy to the cholinergic anti-inflammatory pathway. Neurogastroenterology & Motility. 2013; 25(3):208-21.
40. Cai P Y, Bodhit A, Derequito R, Ansari S, Abukhalil F, Thenkabail S, Ganji S, Saravanapavan P, Shekar C C, Bidari S. Vagus nerve stimulation in ischemic stroke: old wine in a new bottle. Front Neurol. 2014; 5:107.
41. Levine Y A, Koopman F A, Faltys M, Caravaca A, Bendele A, Zitnik R, Vervoordeldonk M J, Tak P P. Neurostimulation of the cholinergic anti-inflammatory pathway ameliorates disease in rat collagen-induced arthritis. PloS one. 2014; 9(8):e104530.
42. Levine Y A, Koopman F, Faltys M, Zitnik R, Tak P-P, editors. Neurostimulation of the cholinergic antiinflammatory pathway in rheumatoid arthritis and inflammatory bowel disease. Bioelectronic Medicine; 2014: Springer.
43. Dawson J, Pierce D, Dixit A, Kimberley T J, Robertson M, Tarver B, Hilmi O, McLean J, Forbes K, Kilgard M P. Safety, feasibility, and efficacy of vagus nerve stimulation paired with upper-limb rehabilitation after ischemic stroke. Stroke. 2016; 47(1):143-50.
44. Guiraud D, Andreu D, Bonnet S, Carrault G, Couderc P, Hagège A, Henry C, Hernandez A, Karam N, Le Rolle V. Vagus nerve stimulation: state of the art of stimulation and recording strategies to address autonomic function neuromodulation. Journal of neural engineering. 2016; 13(4):041002.
45. Koopman F A, Chavan S S, Miljko S, Grazio S, Sokolovic S, Schuurman P R, Mehta A D, Levine Y A, Faltys M, Zitnik R. Vagus nerve stimulation inhibits cytokine production and attenuates disease severity in rheumatoid arthritis. Proceedings of the National Academy of Sciences. 2016; 113(29):8284-9.
46. Pruitt D T, Schmid A N, Kim L J, Abe C M, Trieu J L, Choua C, Hays S A, Kilgard M P, Rennaker R L. Vagus nerve stimulation delivered with motor training enhances recovery of function after traumatic brain injury. Journal of neurotrauma. 2016; 33(9):871-9.
47. Kanashiro A, Bassi G S, Queiróz Cunha Fd, Ulloa L. From neuroimunomodulation to bioelectronic treatment of rheumatoid arthritis. Bioelectronics in medicine. 2018; 1(2):151-65.
48. Levine Y A, Faltys M, Zitnik R. Activation of the inflammatory reflex in rheumatoid arthritis and inflammatory bowel disease; preclinical evidence. Neuromodulation: Elsevier; 2018. p. 1493-502.
49. Alderman M H, Budner N, Cohen H, Lamport B, Ooi W L. Prevalence of drug resistant hypertension. Hypertension. 1988; 11(3 Pt 2):1171-5.
50. Persell S D. Prevalence of resistant hypertension in the United States, 2003-2008. Hypertension. 2011; 57(6):1076-80. doi: 10.1161/HYPERTENSIONAHA.111.170308.
51. Victor R G. Carotid baroreflex activation therapy for resistant hypertension. Nat Rev Cardiol. 2015; 12(8):451-63. doi: 10.1038/nrcardio.2015.96.
52. Available from: https://microtransponder.com/en-gb/stroke/physicians/stroketechnology”.
53. Okun M S, Fernandez H H, Wu S S, Kirsch-Darrow L, Bowers D, Bova F, Suelter M, Jacobson C E, Wang X, Gordon C W, Zeilman P, Romrell J, Martin P, Ward H, Rodriguez R L, Foote K D. Cognition and mood in Parkinson's disease in subthalamic nucleus versus globus pallidus interna deep brain stimulation: the COMPARE trial. Annals of Neurology. 2009; 65(5):586-95. doi: 10.1002/ana.21596.
54. Burdick A P, Fernandez H H, Foote K D. Target specificity for post-DBS anger. Presented at: Movement Disorders. Presented at: Movement Disorders, Movement Disorders Society 14th Annual Congress. 2010.
55. Romo R, Hernández A, Zainos A, Brody C D, Lemus L. Sensing without touching: psychophysical performance based on cortical microstimulation. Neuron. 2000; 26(1):273-8.
56. Lima M C, Fregni F. Motor cortex stimulation for chronic pain Systematic review and meta-analysis of the literature. Neurology. 2008; 70(24):2329-37. doi: 10.1212/01.wnl.0000314649.38527.93.
57. Tsubokawa T, Katayama Y, Yamamoto T, Hirayama T, Koyama S. Chronic Motor Cortex Stimulation for the Treatment of Central Pain. In: Hitchcock PER, Broggi P D G, Burzaco D J, Martin-Rodriguez D J, Meyerson DBA, Tóth D S, editors. Advances in Stereotactic and Functional Neurosurgery 9: Springer Vienna; 1991. p. 137-9.
58. Tsubokawa T, Katayama Y, Yamamoto T, Hirayama T, Koyama S. Chronic motor cortex stimulation in patients with thalamic pain. Journal of Neurosurgery. 1993; 78(3):393-401. doi: 10.3171/jns.1993.78.3.0393.
59. Plautz E J, Barbay S, Frost S B, Friel K M, Dancause N, Zoubina E V, Stowe A M, Quaney B M, Nudo R J. Post-infarct cortical plasticity and behavioral recovery using concurrent cortical stimulation and rehabilitative training: a feasibility study in primates. Neurol Res. 2003; 25(8):801-10. doi: 10.1179/016164103771953880.
60. Johnson B C, Shen K, Piech D, Ghanbari M M, Li K Y, Neely R, Carmena J M, Maharbiz M M, Muller R, editors. StimDust: A 6.5 mm<sup>3</sup>, wireless ultrasonic peripheral nerve stimulator with 82% peak chip efficiency. 2018 IEEE Custom Integrated Circuits Conference (CICC); 2018 8-11 Apr. 2018.
61. Mazzilli F, Lafon C, Dehollain C. A 10.5 cm Ultrasound Link for Deep Implanted Medical Devices. IEEE Transactions on Biomedical Circuits and Systems. 2014; 8(5):738-50. doi: 10.1109/TBCAS.2013.2295403.
62. Charthad J, Chang T C, Liu Z, Sawaby A, Weber M J, Baker S, Gore F, Felt S A, Arbabian A. A mm-Sized Wireless Implantable Device for Electrical Stimulation of Peripheral Nerves. IEEE transactions on biomedical circuits and systems. 2018; 12(2):257-70.
63. Shi C, Andino-Pavlovsky V, Lee S, Costa T, Elloian J, Konofagou E, Shepard K L. Application of a sub-0.1-mm3 implantable mote for in vivo real-time wireless temperature sensing. Science Advances. 2021. Epub May 7, 2021.
64. Llinás R R, Walton K D, Nakao M, Hunter I, Anquetil P A. Neuro-vascular central nervous recording/stimulating system: Using nanotechnology probes. Journal of Nanoparticle Research. 2005; 7(2-3):111-27. doi: 10.1007/s11051-005-3134-4.
65. Kim D-H, Ghaffari R, Lu N, Rogers J A. Flexible and Stretchable Electronics for Biointegrated Devices. Annual Review of Biomedical Engineering. 2012; 14(1):113-28. doi: 10.1146/annurev-bioeng-071811-150018.
66. Rogers J A, Someya T, Huang Y. Materials and Mechanics for Stretchable Electronics. Science. 2010; 327(5973):1603-7. doi: 10.1126/science.1182383.
67. Sun Y, Rogers J A. Inorganic Semiconductors for Flexible Electronics. Advanced Materials. 2007; 19(15):1897-916. doi: 10.1002/adma.200602223.
68. Chang T Y, Yadav V G, De Leo S, Mohedas A, Rajalingam B, Chen C-L, Selvarasah S, Dokmeci M R, Khademhosseini A. Cell and Protein Compatibility of Parylene-C Surfaces. Langmuir. 2007; 23(23):11718-25. doi: 10.1021/la7017049.
69. Weisenberg B A, Mooradian D L. Hemocompatibility of materials used in microelectromechanical systems: Platelet adhesion and morphology in vitro. J Biomed Mater Res. 2002; 60(2):283-91. doi: 10.1002/jbm.10076.
70. Viventi J, Kim D H, Vigeland L, Frechette E S, Blanco J A, Kim Y S, Avrin A E, Tiruvadi V R, Hwang S W, Vanleer A C, Wulsin D F, Davis K, Gelber C E, Palmer L, Van der Spiegel J, Wu J, Xiao J, Huang Y, Contreras D, Rogers J A, Litt B. Flexible, foldable, actively multiplexed, high-density electrode array for mapping brain activity in vivo. Nat Neurosci. 2011; 14(12):1599-605. Epub 2011/11/15. doi: 10.1038/nn.2973. PubMed PMID: 22081157; PubMed Central PMCID: PMCPMC3235709.
71. Shahrjerdi D, Bedell S W, Khakifirooz A, Fogel K, Lauro P, Cheng K, Ott J A, Gaynes M, Sadana D K, editors. Advanced flexible CMOS integrated circuits on plastic enabled by controlled spalling technology. Electron Devices Meeting (IEDM), 2012 IEEE International; 2012 10-13 Dec. 2012.
72. Shahrjerdi D, Bedell S W. Extremely flexible nanoscale ultrathin body silicon integrated circuits on plastic. Nano Lett. 2013; 13(1):315-20. Epub 2012/12/20. doi: 10.1021/n1304310x. PubMed PMID: 23249265.
73. Thimot J, Shepard K L. Bioelectronic devices: Wirelessly powered implants. Nature Biomedical Engineering. 2017; 1:0051.
74. Starr P, Agrawal C, Bailey S. Biocompatibility of common polyimides with human endothelial cells for a cardiovascular microsensor. Journal of Biomedical Materials Research Part A. 2016; 104(2):406-12.
75. Nica S L, Hulubei C, Stoical, Emil Ioanid G, loan S. Surface properties and blood compatibility of some aliphatic/aromatic polyimide blends. Polymer Engineering & Science. 2013; 53(2):263-72.
76. Wei X F, Grill W M. Impedance characteristics of deep brain stimulation electrodes in vitro and in vivo. Journal of neural engineering. 2009; 6(4):046008. doi: 10.1088/1741-2560/6/4/046008.
77. Lempka S F, Miocinovic S, Johnson M D, Vitek J L, McIntyre C C. In vivo impedance spectroscopy of deep brain stimulation electrodes. Journal of Neural Engineering. 2009; 6(4):046001. doi: 10.1088/1741-2560/6/4/046001.
78. Wang M L, Chang T C, Arbabian A, editors. Ultrasonic Implant Localization for Wireless Power Transfer: Active Uplink and Harmonic Backscatter. 2019 IEEE International Ultrasonics Symposium (IUS); 2019 6-9 Oct. 2019.
79. Fenster A, Downey D B, Cardinal H N. Three-dimensional ultrasound imaging. Physics in Medicine and Biology. 2001; 46(5):R67. doi: 10.1088/0031-9155/46/5/201.

1. A vascular neural interface device for at least one of stimulating or recording from the nervous system, comprising:
a package configured to be inserted within a blood vessel, wherein the package includes:
at least one transducer,
at least one electrode, and
at least one integrated circuit,
wherein the at least one transducer is configured to at least one of receive or transmit a wireless signal which is used to at least one of provide energy to or communicate with the at least one integrated circuit to at least one of record information from or stimulate the nervous system using recording electronics or stimulating electronics.

2. The vascular neural interface device of claim 1, wherein the at least one transducer is a piezoelectric transducer configured to interface with ultrasound energy.

3. The vascular neural interface device of claim 1, wherein the package is a flexible circuit board.

4. The vascular neural interface device of claim 3, wherein the package is configured to be deployed with a catheter into the blood vessel by:
rolling the housing around the catheter to form a rolled catheter configuration, and
deploying the rolled catheter configuration at a predetermined location by expanding the catheter configuration against walls of the blood vessel.

5. The vascular neural interface device of claim 3, wherein the at least one flexible circuit board includes polyimide and metal interconnects.

6. The vascular neural interface device of claim 3, wherein the at least one electrode spans fully between opposing sides of the at least one flexible circuit board, such that when unrolled in the blood vessel, the at least one electrode and the at least one flexible circuit board collectively span a circumference of the blood vessel.

7. The vascular neural interface device of paragraph 3, wherein the at least one integrated circuit has a configuration and dimensions to be mechanically flexible.

8. The vascular neural interface device of claim 1, wherein the at least one transducer is configured to facilitate powering and communication with an external device that is rotationally invariant in the blood vessel.

9. The vascular neural interface device of claim 1, further comprising a data transmission arrangement which is configured to transmit data at least one (i) to an external device using an amplitude shift keying procedure, or (ii) from the external device using at least one of the load shift keying procedure or a modulated backscatter procedure.

10. (canceled)

11. The vascular neural interface device of claim 1, wherein at least one chamber is included in the package containing microbubbles.

12. The vascular neural interface device of claim 1, wherein an external device provided outside of a body is mounted on a surface of the vascular neural interface configuration at a particular location for powering and data transmission thereof.

13. The vascular neural interface device of claim 12, wherein the external device is an ultrasound transducer.

14. The vascular neural interface device of claim 13, wherein the ultrasound transducer is a two-dimension array of transducers provided on or in a wearable patch device.

15. (canceled)

16. A method for at least one of stimulating or recording information of a nervous system, comprising:
providing vascular neural interface device which includes a package configured to be inserted within a blood vessel, wherein the package includes:
at least one transducer,
at least one electrode, and
at least one integrated circuit;
with the at least one transducer, at least one of receiving or transmitting a wireless signal; and
at least one of providing energy to or communicating with the at least one integrated circuit to at least one of record information of or stimulate the nervous system using recording electronics or stimulating electronics.

17. The method of claim 16, wherein at least one of:
the at least one transducer is a piezoelectric transducer configured to interface with ultrasound energy,
the package is a flexible circuit board, or
the at least one flexible circuit board includes polyimide and metal interconnects.

18. (canceled)

19. The method of claim 17, further comprising:
deploying the package with a catheter into the blood vessel by:
rolling the package around the catheter to form a rolled catheter configuration, and
deploying the rolled catheter configuration at a predetermined location by expanding the catheter configuration against walls of the blood vessel.

20. The method of claim 17, wherein the at least one flexible circuit board includes polyimide and metal interconnects.

21. The method of claim 17, wherein the at least one electrode spans fully between opposing sides of the at least one flexible circuit board, such that when unrolled in the blood vessel, the at least one electrode and the at least one flexible circuit board collectively span a circumference of the blood vessel.

22. The method of claim 17, wherein the at least one integrated circuit has a configuration and dimensions to be mechanically flexible.

23. The method of claim 16, wherein the at least one transducer is configured to facilitate powering and communication with an external device that is rotationally invariant in the blood vessel.

24. The method of claim 16, further comprising transmitting at least one of:
(a) data at least one of to an external device using an amplitude shift keying procedure, or (ii) from the external device using at least one of the load shift keying procedure or a modulated backscatter procedure, or
(b) signals to locate the device with at least one chamber on the package containing microbubbles.

25-26. (canceled)

27. The method of claim 16, wherein an external device provided outside of a body is mounted on a surface of the vascular neural interface configuration at a particular location for powering and data transmission thereof.

28. The method of claim 27, wherein the external device is an ultrasound transducer.

29. The method of claim 28, wherein the ultrasound transducer is a two-dimension array of transducers provided on or in a wearable patch device.

30. (canceled)
